(19) United States
(12) Patent Application Publication
(10) Pub. No.: US 2023/0097249 A1
(43) Pub. Date: Mar. 30, 2023

(54) IMPRESSION REGISTRATION

(71) Applicant: CLARONAV INC., North York (CA)

(72) Inventors: Doron Dekel, Toronto (CA); Arish Qazi, Oakville (CA)

(21) Appl. No.: 17/947,224

(22) Filed: Sep. 19, 2022

Related U.S. Application Data
(60) Provisional application No. 63/250,273, filed on Sep. 30, 2021.

(51) Int. Cl. A61B 6/03; A61C 9/00

(52) U.S. Cl. CPC A61B 6/032; A61C 9/0006; A61B 2090/3983

(57) ABSTRACT

Systems and methods for providing at least one fixture for use in an image guidance system for determining a registration coordinate mapping between a human jaw and a volumetric image of that jaw. The system includes one or more channeled trays and a moldable thermoplastic material for inserting into a channeled tray. The moldable thermoplastic material is hardenable into an impression part such that when mated to the at least a portion of the human jaw and held within the channel of the channeled tray, the impression part resists displacement relative to the human jaw. At least one channeled tray in the system includes a fiducial region detectable in the volumetric CT image in a fixed spatial relation relative to the coupling surface region and at least one channeled tray is pose-trackable by the image guidance system.

### PRIORITY

This application claims the benefit of U.S. Provisional Patent Application No. 63/250,273, filed Sep. 30, 2021, which is incorporated herein by reference in its entirety.

### FIELD

The described embodiments relate to the field of medical devices, in particular, the field of dental navigation systems.

### INTRODUCTION

Fiducials-based image-to-jaw registration methods applied in dental instrument guidance systems often require highly repeatable, rigid, coupling of the rigid part holding the fiducials to the jaw. Molding a rigid material to the jaw carries a risk of the rigid material getting “locked” on the jaw due to undercuts and spaces between teeth. Using an elastic material to prevent the locking risk enables an undesirable movement of the part relative to the jaw, putting repeatability at risk. These conflicting requirements can make it challenging to find a repeatable jaw coupling design for registration.

### SUMMARY

The various embodiments described herein generally relate to systems and methods for providing at least one fixture for an image-based guidance system for determining a registration coordinate mapping that aligns a human jaw with its volumetric CT (computerized X-ray tomography) image. An example kit includes:
one or more channeled trays made of a rigid material that will not substantially deform when subjected to temperatures of about 70 degrees C. (Celsius), a portion of the surface of the channel in each channeled tray defining a coupling surface region;
a moldable thermoplastic material for inserting into a channeled tray and for molding into a molded shape within the channeled tray, wherein

the molded shape of the moldable thermoplastic material mates with a surface geometry of at least a portion of the human jaw and the coupling surface region of the channeled tray;

the moldable thermoplastic material is hardenable into an impression part such that when mated to the at least a portion of the human jaw and held within the channel of the channeled tray, the impression part resists displacement relative to the human jaw;

the channeled tray is removable from the impression part;

each channeled trays of the one or more channeled trays is individually repeatedly attachable to the impression part;

when a channeled tray is removed from the impression when the impression part is mated to the at least a portion of the human jaw, the impression part is bendable away from the surface geometry of the at least a portion of the human jaw such that the impression part is removable from, and repeatedly attachable to, the portion of the human jaw;

at least one channeled tray comprises a fiducial region detectable in the volumetric CT image in a fixed spatial relation relative to the coupling surface region; and

at least one channeled tray is pose-trackable by the image guidance system.

In any embodiment, the moldable thermoplastic material may be provided as a sheet less than 4 mm in thickness and 10 g in weight.

In any embodiment, the moldable thermoplastic material may be provided as a sheet less than 2 mm in thickness and 4 g in weight.

In any embodiment, the sheet may become moldable when heated to a temperature in the range of 50-90° C.

In any embodiment, the sheet may be heatable by hot air at an air temperature of about 200° C. for about 1 minute.

In any embodiment, the kit may further comprise a hot air heater for heating the sheet to the temperature in the range of 50-90° C.

In any embodiment, the fiducial region may comprise a plurality of cylindrical fiducials of at least two different diameters

In any embodiment, each channeled tray may comprise a base, a lingual wall and a facial wall, the lingual wall and facial wall extending from the base and spaced from each other so as to be adapted to receive at least one tooth, and wherein the fiducial region comprises a plurality of cylindrical fiducials embedded in the base of at least one of the channeled trays.

In any embodiment, the kit may comprise two optically pose-trackable channeled trays, each having trackable optical markings, wherein the trackable optical markings on each of the two trays may be in substantially different poses relative to the coupling surface region.

In any embodiment, the coupling surface region shared by each channeled tray may be shaped to constrain the motion for removing the tray from the impression part such that when attached to the impression part, the channeled tray remains in a fixed spatial relationship to the impression part when not pulled away from it by a human operator.

In any embodiment, a pose-trackable channeled tray may carry trackable optical markings, and the kit may further comprise calibration data for defining a mapping between a coordinate frame of the trackable optical markings and a coordinate frame of the impression part when the channeled tray is in the fixed spatial relationship to the impression part.

In any embodiment, the coupling surface region may be shaped to prevent full insertion of the impression part into the channel in a channeled tray in an orientation or position other than a single unique one.

In any embodiment, the kit may comprise two channeled trays, wherein one of the two trays may be pose-trackable, and wherein a portion of the channel in the pose-trackable tray may be narrower than the corresponding portion of the channel in the other tray to provide it with increased retention when coupled to an impression part molded by the other tray.

An example method for determining a registration coordinate mapping between a human jaw and a volumetric CT (computerized X-ray tomography) image of that jaw includes:
providing one or more channeled trays made of a rigid material that will not substantially deform when subjected to temperatures of about 70 degrees C., a portion of the surface of the channel in each channeled tray defining a coupling surface region;
providing a moldable thermoplastic material;
inserting the moldable thermoplastic material into a channeled tray comprising a fiducial region in a fixed spatial relation relative to the coupling surface region, the boundary of the fiducial region being detectable in a volumetric CT image;
forming a molded shape of the moldable thermoplastic material in the channeled tray to mate with a surface geometry of at least a portion of the human jaw and the coupling surface region of the channeled tray;
hardening the molded shape of the moldable thermoplastic material into an impression part such that i) when mated to the at least a portion of the human jaw and held within a channel of a channeled tray, the impression part resists displacement relative to the human jaw ii) the channeled tray is removable from the impression part, iii) each channeled trays of the one or more channeled trays is individually repeatedly attachable to the impression part, and iv) the impression part is removable from, and repeatedly attachable to, the portion of the human jaw;
obtaining a volumetric CT image of a portion of the jaw and the channeled tray coupled to it by the impression part;
removing the channeled tray from the impression part;
bending the impression part away from the surface geometry of the at least a portion of the human jaw to remove the impression part from the human jaw; and subsequently mating the impression part with the surface geometry of the at least a portion of the human jaw; then
coupling a trackable channeled tray that is pose-trackable by the image guidance system to the impression part; and
determining a registration coordinate mapping between the human jaw and its volumetric CT image based on a coordinate mapping between a coordinate frame of the pose-trackable tray and a coordinate frame of the fiducial region's appearance in the image.

In any embodiment, the method may further comprise providing calibration data defining a mapping between a coordinate frame of the trackable channeled tray and a coordinate frame of the fiducial region, and wherein determining the registration coordinate mapping between the human jaw and its volumetric CT image may be partly based on the calibration data.

In any embodiment, the method may further comprise determining a selected position in the coordinate frame of the pose-trackable tray, and then using the registration coordinate mapping to map coordinates of the selected position to a corresponding position in the volumetric CT image.

In any embodiment, each channeled tray may define a corresponding channel comprising a base, a lingual wall and a facial wall, the lingual wall and facial wall extending from the base and spaced from each other so as to be adapted to receive at least one tooth; and
forming the molded shape of the moldable thermoplastic material in the channeled tray may comprise placing the channeled tray relative to the jaw such that at least one tooth is received within the corresponding channel of the channeled tray to mold the moldable thermoplastic material therein.

In any embodiment, the method may further comprise molding some of the moldable thermoplastic material over nearby surfaces of the human jaw, the nearby surfaces of the human jaw being outside the corresponding channel of the channeled tray.

In any embodiment, providing the moldable thermoplastic material may comprise providing the moldable thermoplastic material as a sheet less than 4 mm in thickness and 10 g in weight

In any embodiment, providing the moldable thermoplastic material may comprise providing the moldable thermoplastic material as a sheet less than 2 mm in thickness and 4 g in weight.

In any embodiment, the method may further comprise before forming the molded shape of the moldable thermoplastic material in the channeled tray, softening the sheet by heating the sheet to a temperature in the range of 50-90° C.

In any embodiment, the method may further comprise before forming the molded shape of the moldable thermoplastic material in the channeled tray, heating the sheet to a temperature in the range of 50-90° C., to soften the sheet, by blowing air at a temperature higher than 100° C. over a surface the sheet.

In any embodiment, each channeled tray may define a corresponding channel comprising a base, a lingual wall and a facial wall, the lingual wall and facial wall extending from the base and spaced from each other so as to be adapted to receive at least one tooth; and,
inserting the moldable thermoplastic material into the channeled tray comprises folding or rolling the sheet before inserting into the corresponding channel of the channeled tray.

These and other aspects and features of various embodiments will be described in greater detail below.

### BRIEF DESCRIPTION OF THE DRAWINGS

Several embodiments will now be described in detail with reference to the drawings, in which:

FIG. 1 is an example illustration of a registration system, according to at least one embodiment;

FIGS. 2A-2D are example illustrations of a tray, according to at least one embodiment;

FIGS. 3A-3B are example images of a moldable thermoplastic material molded into an impression part, according to at least one embodiment;

FIG. 4 is an example illustration of a pose-trackable tray with three trackable arms overlapping at the tray, according at least one embodiment;

FIG. 5 is an example illustration of a method of heating and molding a moldable thermoplastic material, according to at least one embodiment;

FIG. 6 is an example illustration of a CT scanner and impression part storage, according to at least one embodiment;

FIG. 7 is an example illustration of a pose-trackable tray and impression part, according to at least one embodiment; and

FIGS. 8-9 are flowcharts of an example method of providing at least one fixture for an image guidance system for determining and mapping a pose of a selective position on a human jaw to a corresponding position in a volumetric CT image.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that the term “coupled” used herein indicates that two elements can be directly coupled to one another or coupled to one another through one or more intermediate elements.

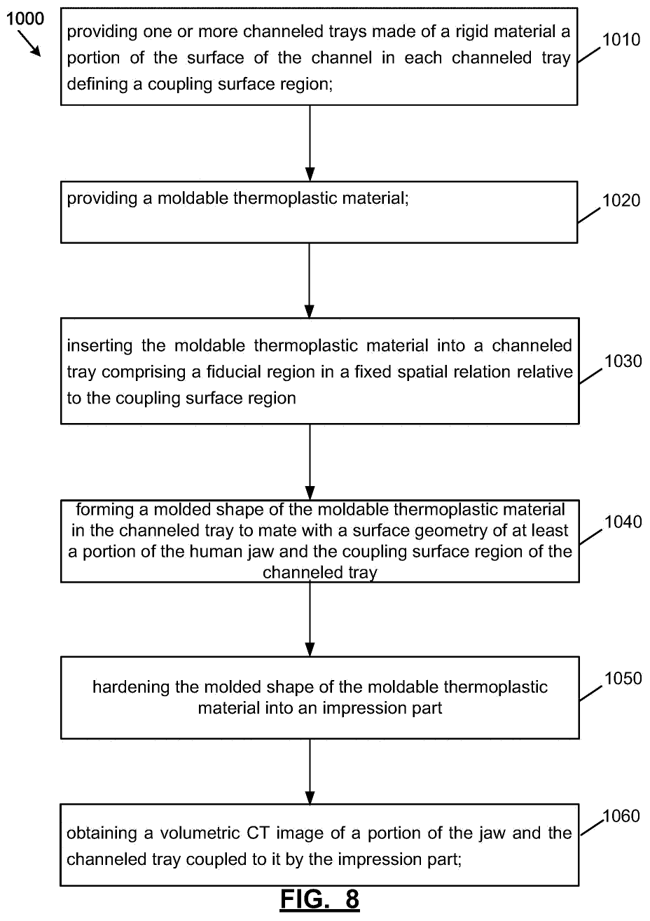

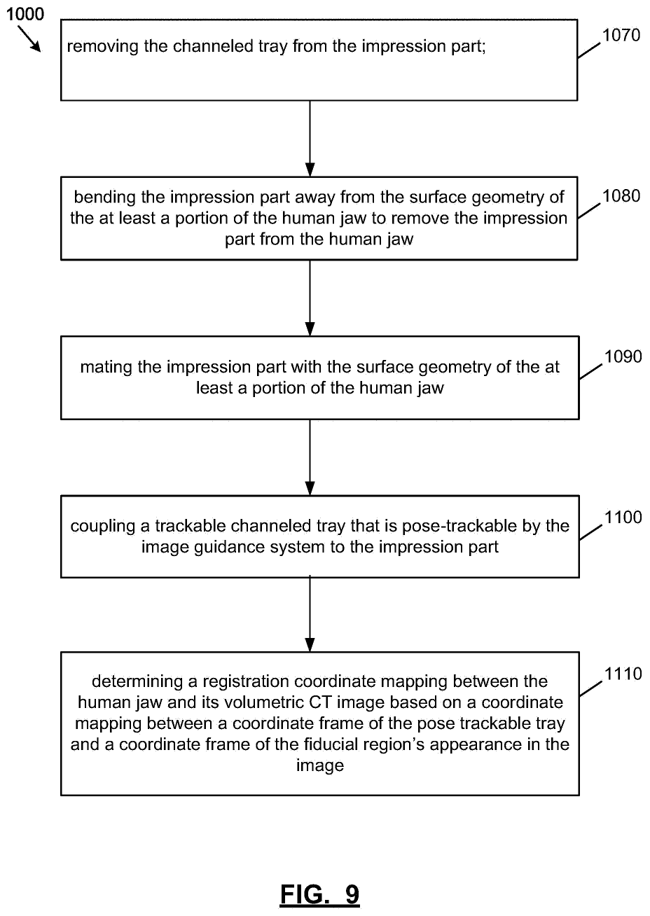

In at least one embodiment, aspects of methods described herein, such as method 1000 described with reference to FIGS. 8 and 9 below, may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication component. For example, and without limitation, the programmable computer (referred to below as data processor) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In at least one embodiment, the communication component may be a network communication interface. In embodiments in which elements are combined, the communication component may be a software communication interface, such as those for inter-process communication (IPC). In still other embodiments, there may be a combination of communication components implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g., ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

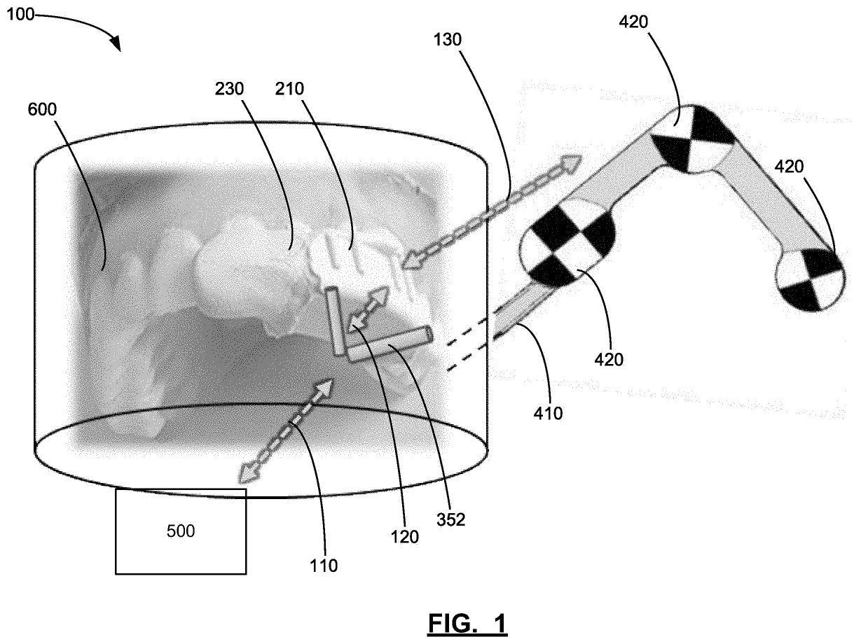

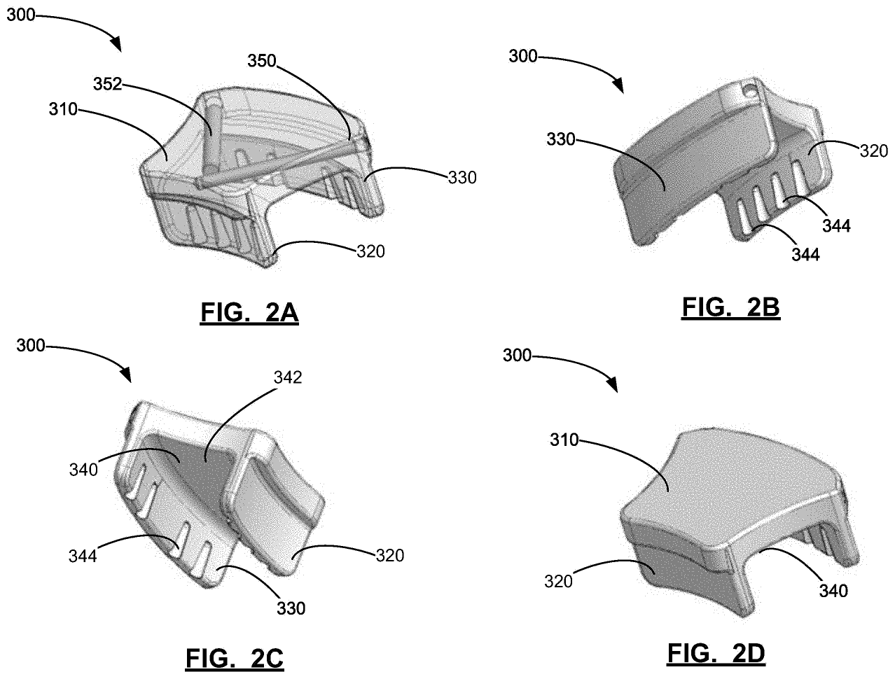

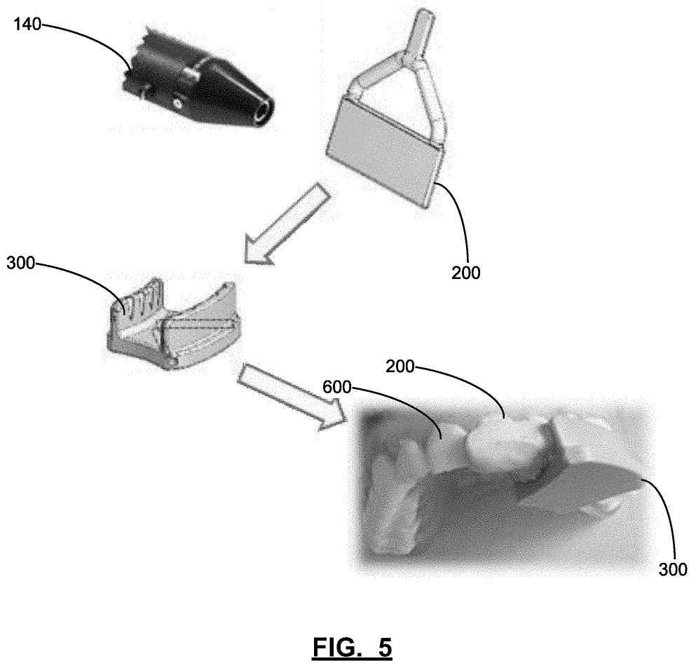

Referring now to FIG. 1, shown therein is an example illustration of a system 100 of at least one fixture for an image guidance system for determining a registration coordinate mapping for mapping selected positions on or within a human jaw 600 and corresponding positions in a volumetric CT (computerized X-ray tomography) image 500 of that jaw 600, according to at least one embodiment. This system 100 may also be referred to as an impression registration system. The system 100 includes a moldable thermoplastic material 200 (shown in FIG. 5) and a tray 300 (FIG. 2).

The impression registration system 100 may be used to register a coordinate system of a trackable object 420 rigidly attached jaw 600 with the coordinate system of its CT image 500 to accurately align the anatomical features of the jaw with their appearance in the image. Registering the jaw 600 with the CT image 500 may allow pose-trackable components of the system 100 to be used for providing guidance during the treatment of a patient. The system 100 may be provided as a kit of various components, depending on the desired use of the system 100.

Referring to FIG. 2, shown therein is an example tray 300. The tray 300 may have a base 310, a lingual wall 320, and a facial wall 330. The lingual wall 320 and the facial wall 330 may extend from the base 310 and may be spaced apart from each other so as to be adapted to receive at least one tooth. The tray 300 includes a channel 340 with a portion of the surface of the channel 340 defining a coupling surface region 342. Each tray in the system 100 is made of a rigid material that may not substantially deform when subjected to a temperature of about 70 degrees C. Maintaining the rigidity of the tray 300 may allow for the insertion of the moldable thermoplastic material 200 at higher temperatures without deforming the tray 300.

The moldable thermoplastic material 200 is insertable into the tray 300 to mold into a molded shape within the channeled tray. The molded shape of the moldable thermoplastic material 200 mates with a surface geometry of at least a portion of the human jaw 600 and the coupling surface region 342 of the tray 300. The moldable thermoplastic material 200 is then hardenable into an impression part 210 such that when mated to the at least a portion of the human jaw and held within the channel 340 of the tray 300, the impression part 210 resists displacement relative to the human jaw. Resisting displacement relative to the jaw may allow for repeatable measurements to be taken during the registration and the subsequent guidance processes, thereby improving the accuracy of the mapping and subsequent use during treatment.

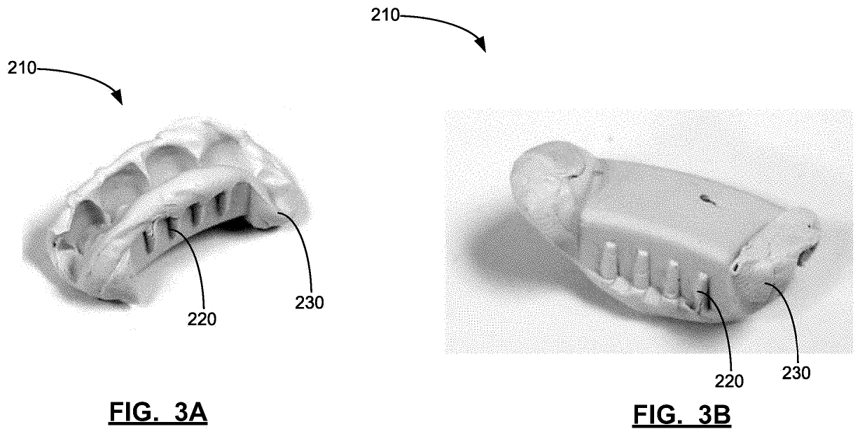

The moldable plastic material 200 may have sufficient viscosity, when heated, that it does not penetrate deeply into cracks between the teeth of the patient. Excess material 230, as shown in FIGS. 1 and 3, may be ejected from the tray 300 during the molding process. This excess material 230 may be molded over nearby surfaces to increase the stability and retention of the material 200 to the jaw of the patient. As shown in FIG. 1, the excess material 230 has been finger-molded over adjacent teeth. Freehand molding may allow for the simple addition of stability and retention without the need for additional tools. This additional stability and retention provided by the excess material 230 may be helpful especially when the teeth being coupled to lack any undercuts, such as incisors.

Once the impression part 210 is formed by hardening, the tray 300 is removable from the impression part 210. Each tray used in the system 100 may be individually repeatably attachable to the impression part 210. When the impression part 210 is mated to the at least a portion of the human jaw, the tray 300 may be removed from the impression part 210 by pulling it away. The impression part 210 is then free to be bent away and removed from the surface geometry of the at least a portion of the human jaw. Thus, the impression part 210 is removable from, and repeatedly attachable to, the portion of the human jaw.

The coupling surface region 342 in each channeled tray 300 may be shaped to constrain the motion for removing the tray 300 from the impression part 210 such that when the tray 300 is attached to the impression part 210, the tray remains in a fixed spatial relationship to the impression part 210 when not pulled away from it by a human operator. In some embodiments, the coupling surface region 342 may be shaped to prevent full insertion of the impression part 210 into the channel 340 in an orientation or position other than a single unique one. In other words, the impression part 210 may only be received by the coupling surface region 342 in a single orientation and/or position. For example, referring to FIGS. 2A-2D and 3A-3B, the tray 300 includes a plurality of recesses 344 that receive protrusions 220 in the impression part 210. The protrusions 220 were formed by inserting the moldable thermoplastic material 200 into the tray 300 such that the material 200 at least partially filled the plurality of recesses 344. When the impression part 210 is inserted into the tray 300, the protrusions 220 limit the positioning of the impression part 210 in the tray aside from an orientation or position other than the unique position in which the recesses 344 are mated with the protrusions 220.

The shape of the tray 300 may be designed such that the tray 300 is removable from the hardened impression part 210 by pulling the tray 300 away from the impression part 210. The tray's position may be restored relative to the hardened impression part 210 by pushing the tray 300 back onto the impression part 210. When hardened, the impression part 210 may have sufficient elasticity to be bent as needed to release it from the jaw, thereby releasing the impression part 210 from any undercuts. Thus, the impression part 210 may form a molded part that can be removed from the jaw while still providing sufficient rigidity to the tray 300 such that the tray 300 may be used for tracking during treatment.

The moldable thermoplastic material 200 may be provided as a sheet. For example, referring to FIG. 5, a sheet of moldable thermoplastic material 200 is gripped by a handle so that the material 200 may be heated to soften it for the molding process. Providing the moldable thermoplastic material 200 as a sheet enables heat applied at its surface, for example by blowing hot air, to rapidly be absorbed and propagate to soften the thermoplastic material volume needed for molding. For example, the sheet may be less than about 4 mm thick, optionally less than about 2 mm thick. The moldable thermoplastic material 200 may range in weight. For example, the sheet of moldable thermoplastic material 200 may be less than about 10 g, optionally less than about 4 g in weight. The use of thin sheets of moldable plastic material 200 may reduce the time and cost associated with preparation of the impression part 210.

The moldable thermoplastic material may be heated to facilitate the molding process. For example, the moldable thermoplastic material 200 may become moldable when heated to a temperature in the range of 50-90° C. The moldable thermoplastic material 200 may be heated by any means, including, but not limited to hot air. For example, the material 200 may be heatable by hot air at an air temperature of about 200° C. for about 1 minute to become moldable. In some embodiments, the system 100 may include a hot air heater 140 for heating the moldable thermoplastic material 200 to the range of 50-90° C. The use of hot air instead of water may increase the speed of heating and reduce mess associated with the heating process.

The moldable plastic material 200 may be any material that is viscous enough when heated to mold to at least a portion of a human jaw. For example, the material may be viscous enough to be pressed around teeth of the patient such that the material molds to the teeth. The moldable plastic material 200 may be, including, but not limited to, a compound based on polycaprolactone and/or a biodegradable polyester such as Rolyan Polyform.

The tray 300 may include a fiducial region 350 detectable in the volumetric CT image in a fixed spatial relation relative to the coupling surface 342. The fiducial region 350 may include one or more fiducials 352 embedded in the base 310 of the tray 300. For example, as shown in FIG. 2A, the fiducial 352 may be cylindrical, such as a pin-shape.

The fiducials 352 may be arranged in a manner that is not rotationally symmetrical. A rotationally asymmetrical arrangement can be used to uniquely define a coordinate frame anchored to the tray 300. As described above, the tray 300 may be uniquely positionally coupled to the impression part 210 when mated to the impression part 210 and may remain anchored during the CT scan.

The fiducials 352 may be made of a material with radiopacity similar to dense bone. For example, the fiducials 352 may be made of an aluminum alloy. In contrast, the tray 300 may be made of a material that has a similar density to soft tissue, such as a thermoplastic. Using different materials may improve the ability of the imaging system to discern the difference between the tray 300 and the fiducials. The different radiopacities of the materials used in the system 100 may also allow for automatic calibration of image values in the CT scan.

The cylindrical fiducials 352 may have the same diameter or may have at least two different diameters. For example, two diameters may be 1.5 mm and 2.0 mm. The size of the fiducials 352 may vary depending on the use of the system 100. Using fiducials 352 with two or more different diameters may improve the reliability of automatic detection and localization of the fiducials 352 in the CT image, facilitating distinguishing fiducials 352 from one another. As shown in FIG. 2, the fiducials 352 can be embedded in the tray 300 such that when they are imaged in the CT, they are in the occlusion, between the two jaws. This positioning may reduce the likelihood of radial metal artifacts in the CT image obscuring their boundary. In other words, the positioning of the fiducials 352 may reduce the likelihood of the appearance of the tooth crowns and bone in CT image 500 being corrupted by metal artifacts. Positioning the fiducials 352 adjacent to the treatment site may also improve the likelihood of the fiducials 352 being present in the CT scan field of view.

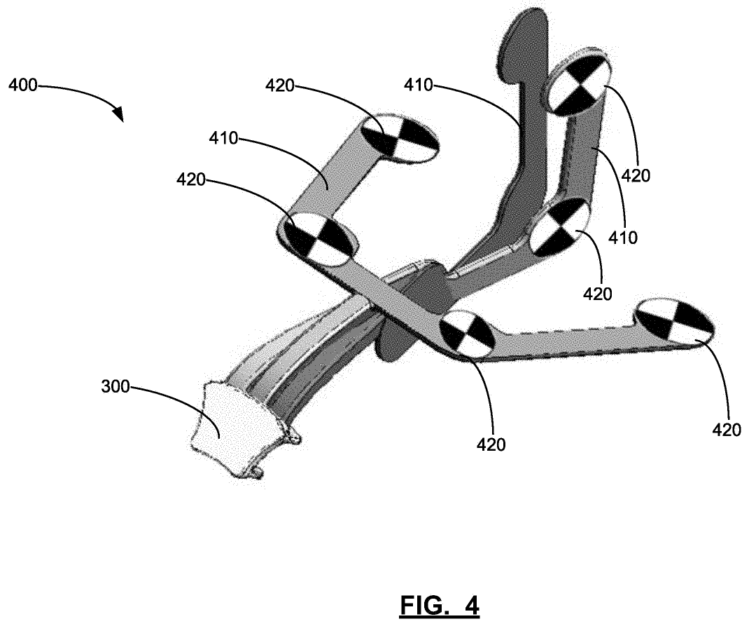

The system 100 can include a plurality of trays 300. Each tray 300 in the system 100 may be used for the same or different purposes. For example, the system 100 may include at least one pose-trackable tray 400 that is pose-trackable by an image guidance system. The pose-trackable tray 400 may have the same or different structure than the tray 300. Referring to FIG. 4, the tray 400 illustrated therein includes a portion having the same structure as the tray 300 described previously. The tray 400 also includes a trackable arm 410 and is pose-trackable by trackable optical markings 420 positioned on the trackable arm 410. The trackable arm 410 is coupled to the tray 300.

The pose-trackable tray 400 may be positioned over the impression part 210 to secure the pose-trackable tray 400 to the jaw 600. This coupling between the pose-trackable tray 400 and the jaw 600 may allow for tracking of the jaw during the guidance process. The coordinate mapping between the pose-trackable tray 400 and its respective trackable optical markings 420 and the fiducials 352 present in the CT scan may be pre-calibrated to provide registration without the need for calibration or fiducial localization prior to guidance. This registration may be relatively instantaneous.

The system 100 may include calibration data for defining a mapping between a coordinate frame of the trackable optical markings 420 and a coordinate frame of the impression part when the tray 400 is in the fixed spatial relationship to the impression part.

An example coordinate system may be defined by the centerlines of two co-planar pin fiducials. The intersection point between the centerlines may define the origin. The centerline of the thicker pin (larger diameter) may define the x-axis and the cross product between the centerline vectors may define the z-axis. The y-axis may be defined as the cross product of the x and z-axis vectors.

The system 100 may include a plurality of pose-trackable trays 400 with each tray 400 having trackable optical markings 420 facing the camera when the tray is mounted at different positions along the jaw arch. For example, as shown in FIG. 4, the tray 400 may be provided with three different variations of a trackable arm 410 each having the optical markings 420 facing in a direction suitable for mounting the tray in the left, center and right parts of each jaw. FIG. 4 shows all three variations overlapping at the tray to illustrate the different arm position and orientations. The three variations may be used to track the jaw when the tray is mounted at sextants 1 and 4, 2 and 5, and 3 and 6, respectively while the markings 420 face towards the tracking camera in front of the face, and do not interfere with a surgeon's motions during surgery when the tray 400 is attached to the jaw in its designated sextant.

Computing the mapping between the pose-tracking space and the CT space may involve three mappings, as illustrated in FIG. 1. The first mapping 110 may be computed between the CT space and the fiducial space and may be automatically computed by software when the CT image is imported. Automatically computing the mapping may involve automatic detection of the fiducials 352 using their size and shape characteristics, localizing their centerlines in image coordinates, and using the centerline parameters to derive the mapping between the fiducial coordinate spate and the CT image coordinates. These steps may use image processing algorithms, such as, including, but not limited to, tube model fitting.

The second mapping 120, between the fiducials 352 and the impression part 210 may be determined by the shape of the tray 300. The tray 300 may be measured once using a specialized jig that couples a trackable marker defining a trackable coordinate system to the impression part 210. The tray 300 may then be mounted to the impression part 210 and the centerlines of the two pin boreholes in the tray 300 in the impression part 210 coordinate system may be measured (as defined by the trackable marker).

The third mapping 130 may be measured for each pose-trackable tray 400 using the specialized jig described above and may be communicated to the navigation system software using an optical tracking marker on the pose-trackable tray 400. Each trackable arm 410 may be individually calibrated during the production process by measuring the mapping between the coordinate system of the trackable optical markings 420 and the coordinate system of the impression part 210. The appropriate calibration information may be retrieved by the navigation software using the trackable optical markings 420 on each trackable arm 410 as a part identifier. The part identifier can be registered prior to mapping so that the navigation software is able to calibrate each trackable arm 410. Pre-calibrating the pose-trackable tray 400 may improve the accuracy of tracking while eliminating the need for the user to perform a time-consuming tracker calibration prior to the guidance process, thereby simplifying and speeding up the guidance process.

In some embodiments, the pose-trackable tray 400 may have a portion of the channel that is narrower than the corresponding portion of the channel in the tray 300 to provide the narrowed channel with increased retention when coupled to the impression part 210 molded by the tray 300.

Method for Determining Registration Coordinate Mapping Between a Human Jaw and a Volumetric CT Image of that Jaw

Referring to FIGS. 5-7, shown therein is an example workflow using the system 100 described herein. Referring to FIG. 5, prior to the CT scan, the moldable plastic material 200 is heated to a temperature of approximately 70° C. using a hot air heater 140. For rapid heating of the material 200 while reducing the risk of overheating, the hot air heater may blow air at a temperature of about 200° C. As shown, the moldable plastic material 200 may begin as a sheet. The sheet may optionally have a thickness of about 1.5 mm to about 2 mm and may weigh approximately 3-4 g. The sheet of moldable plastic material 200 may be heated for approximately 1 minute using the hot air until it becomes soft and/or less rigid. The soft moldable thermoplastic material 200 may then be torn away from the (non-heated) handle, folded and/or rolled and inserted into the channel 340 of tray 300.

The tray 300 containing the heated moldable plastic material 200 can then be pressed against at least a portion of the jaw 600 of the patient, either by an operator (such as dentist or dental assistant), or by having the patient bite on the tray. As shown, the tray 300 is pressed against approximately three teeth neighbouring the treatment site. The tray 300 may be left for approximately one minute to allow the moldable plastic material 200 to harden into the impression part 210. For example, leaving the previously heated moldable thermoplastic material 200 for one minute allows the material to cool, thereby hardening into the impression part 210. In some embodiments, the moldable material 200 may be hardened by a chemical reaction. For example, the moldable material 200 may be hardened by a chemical reaction.

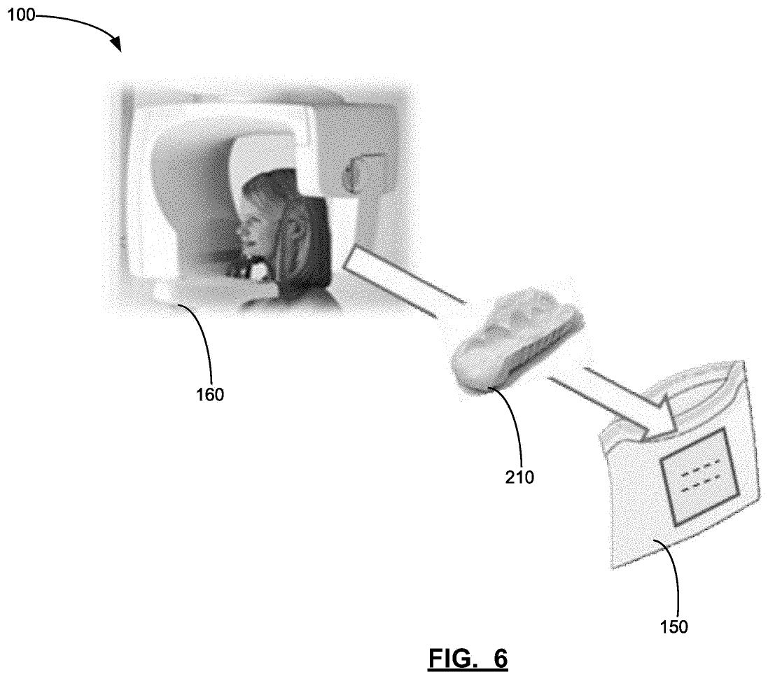

Referring to FIG. 6, the tray 300 containing the impression part 210 is left in place against the patient's jaw 600 for the CT scan using a CT scanner 160. During the CT scan, the fiducials 352 may be identified and segmented. The voxel values in the CT image can then be automatically mapped to standardized Hounsfield Units (HU) of radiopacity using the known HU values of the materials from which the fiducials 352 and the surrounding material of the tray 300 are made of. A linear mapping may be formed between the CT image values and the HU values, which may improve the diagnostic value of the CT image and provide useful information for pre-surgical planning. For example, HU can indicate bone density and therefore the expected stability of implants inserted into the surgical region.

Once the CT scan is complete, the tray 300 may be removed from the impression part 210. The impression part 210 may then be removed from the teeth. As described previously, the impression part 210 may be separable from the tray 300 due to the design of the channel 340 to grip the impression part 210 while still allowing it to be removed by the user by pulling it away in a specific direction. Without the constraining tray, the plastic impression part 210 may be flexible enough to be removable from the jaw 600 of the patient even with the presence of undercuts. Once removed, the tray 300 may be disinfected for reuse, and the impression part 210 may be stored, for example, in a bag 150, for future use.

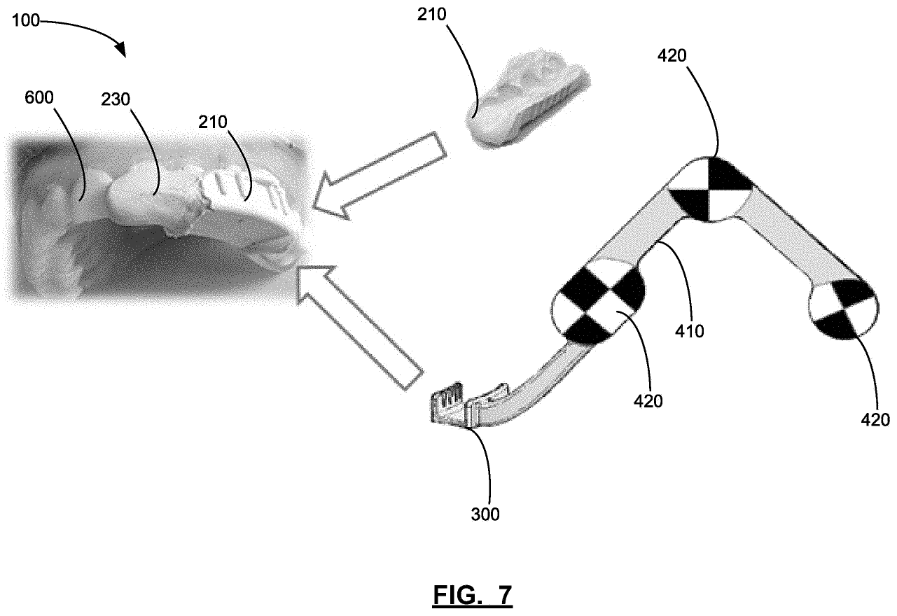

Referring to FIG. 7, the impression part 210 can be reattached to the portion of the jaw 600 to which it was initially molded to begin the treatment of the patient. A pose-trackable tray 300 can be mounted to the impression part 210, held in place by friction. The registration of the pose-trackable tray 300 to the jaw 600 may then be completed, allowing for the beginning of the guidance process during treatment.

Referring to FIGS. 8 and 9, shown therein is an example flow chart of a method 1000 for determining registration coordinate mapping between a human jaw and a volumetric CT image of that jaw.

At 1010, one or more channeled trays 300 are provided. The channeled trays are made of a rigid material that will not substantially deform when subjected to temperatures of about 70 degrees C. A portion of the surface of the channel 340 in each channeled tray 300 defines a coupling surface region 342.

At 1020, a moldable thermoplastic material 200 is provided. Optionally, the moldable thermoplastic material 200 may be softened by heating the material 200 to a temperature in the range of 50-90° C. As described previously, the heating process may use hot air, e.g., air at a temperature higher than 100° C., passing over a surface of the moldable thermoplastic material 200.

At 1030, the moldable thermoplastic material 200 is inserted into the channeled tray. Optionally, inserting the moldable thermoplastic material 200 into the tray 300 may include folding or rolling the material 200 prior to inserting it into the channel 340 of the tray 300. The channeled tray 300 has a fiducial region 350 in a fixed spatial relation relative to the coupling surface region with the boundary of the fiducial region 350 being detectable in a volumetric CT image.

At 1040, a molded shape of the moldable thermoplastic material 200 is formed in the channeled tray 300 to mate with a surface geometry of at least a portion of the human jaw 600 and the coupling surface 342 of the channeled tray 300. For example, the channel 340 in the channeled tray 300 may be sized and shaped such that it is adapted to receive at least one tooth and the molded shape formed by the moldable thermoplastic material 200 may be formed by receiving at least one tooth in the channel 340 of the tray 300. Optionally, some of the moldable thermoplastic material 200 may be molded over nearby surfaces of the human jaw 600 that are outside the corresponding channel 340 of the channeled tray 300.

At 1050, the molded shape of the moldable thermoplastic material 200 is hardened into an impression part 210 such that when mated to the at least a portion of the human jaw 600 and held within a channel 340 of the channeled tray 300, the impression part 210 resists displacement relative to the human jaw 600. The channeled tray 300 is removable from the impression part 210 and each channeled tray 300 is individually repeatedly attachable to the impression part 210. The impression part 210 is removable from, and repeatedly attachable to, the portion of the human jaw 600.

At 1060, a volumetric CT image 500 of a portion of the jaw 600 and the channeled tray 300 coupled to it by the impression part 210 is obtained.

At 1070, the channeled tray 300 is removed from the impression part 210.

At 1080, the impression part 210 is bent away from the surface geometry of the at least a portion of the human jaw 600 to remove the impression part 210 from the human jaw 600.

At 1090, the impression part 210 is again mated with the surface geometry of the at least a portion of the human jaw 600.

In some embodiments, when the CT scan is taken just prior to the dental procedure, impression part 210 may be left on the jaw between the scan and the procedure, removing the need to perform steps 1080 and 1090.

At 1100, a trackable channeled tray 400 that is pose-trackable by the image guidance system is coupled to the impression part 210.

At 1110, a registration coordinate mapping between the human jaw and its volumetric CT image is determined based on a coordinate mapping between a coordinate frame of the pose-trackable tray 400 and a coordinate frame of the fiducial region's appearance in the image. Optionally, calibration data may be provided defining a mapping between a coordinate frame of the pose-trackable tray 400 and a coordinate frame of the fiducial region. Determining the registration coordinate mapping between the human jaw 600 and the volumetric CT image may be at least partly based on the calibration data.

In some embodiments, the method 1000 may further include determining a selected position in the coordinate frame of the pose-trackable tray 400 and using the registration coordinate mapping to map coordinates of the selected position to a corresponding position in the volumetric CT image.

Various embodiments have been described herein by way of example only. Various modification and variations may be made to these example embodiments without departing from the spirit and scope of the invention, which is limited only by the appended claims.

1. A kit for providing at least one fixture for use in an image guidance system for determining a registration coordinate mapping between a human jaw and a volumetric image of that jaw, the kit comprising:
one or more channeled trays made of a rigid material that will not substantially deform when subjected to temperatures of about 70 degrees C., a portion of the surface of the channel in each channeled tray defining a coupling surface region;
a moldable thermoplastic material for inserting into a channeled tray and for molding into a molded shape within the channeled tray, wherein
the molded shape of the moldable thermoplastic material mates with a surface geometry of at least a portion of the human jaw and the coupling surface region of the channeled tray;
the moldable thermoplastic material is hardenable into an impression part such that when mated to the at least a portion of the human jaw and held within the channel of the channeled tray, the impression part resists displacement relative to the human jaw;
the channeled tray is removable from the impression part;
each channeled trays of the one or more channeled trays is individually repeatedly attachable to the impression part;
when a channeled tray is removed from the impression when the impression part is mated to the at least a portion of the human jaw, the impression part is bendable away from the surface geometry of the at least a portion of the human jaw such that the impression part is removable from, and repeatedly attachable to, the portion of the human jaw;
at least one channeled tray comprises a fiducial region detectable in the volumetric CT image in a fixed spatial relation relative to the coupling surface region; and
at least one channeled tray is pose-trackable by the image guidance system.

2. The kit as defined in claim 1 wherein the moldable thermoplastic material is provided as a sheet less than 4 mm in thickness and 10 g in weight.

3. The kit as defined in claim 1 wherein the moldable thermoplastic material is provided as a sheet less than 2 mm in thickness and 4 g in weight.

4. The kit as defined in claims 2 wherein the sheet becomes moldable when heated to a temperature in the range of 50-90° C.

5. The kit as defined in claims 2 wherein the sheet becomes moldable when heated by hot air at an air temperature of about 200° C. for about 1 minute.

6. The kit as defined in claim 4 wherein the kit further comprises a hot air heater for heating the sheet to the temperature in the range of 50-90° C.

7. The kit as defined in claim 1 wherein the fiducial region comprises a plurality of cylindrical fiducials of at least two different diameters

8. The kit as defined in claim 1 wherein each channeled tray comprises a base, a lingual wall and a facial wall, the lingual wall and facial wall extending from the base and spaced from each other so as to be adapted to receive at least one tooth, and wherein the fiducial region comprises a plurality of cylindrical fiducials embedded in the base of at least one of the channeled trays.

9. The kit as defined in claim 1 comprising two optically pose-trackable channeled trays, each having trackable optical markings, wherein the trackable optical markings on each of the two trays are in substantially different poses relative to the coupling surface region.

10. The kit as defined in claim 1 wherein the coupling surface region shared by each channeled tray is shaped to constrain the motion for removing the tray from the impression part such that when attached to the impression part, the channeled tray remains in a fixed spatial relationship to the impression part when not pulled away from it by a human operator.

11. The kit as defined in claim 1 wherein a pose-trackable channeled tray carries trackable optical markings, and the kit further comprises calibration data for defining a mapping between a coordinate frame of the trackable optical markings and a coordinate frame of the impression part when the channeled tray is in the fixed spatial relationship to the impression part.

12. The kit as defined in claim 1, wherein the coupling surface region is shaped to prevent full insertion of the impression part into the channel in a channeled tray in an orientation or position other than a single unique one.

13. The kit as defined in claim 1 comprising two channeled trays, wherein one of the two trays is pose-trackable, and wherein a portion of the channel in the pose-trackable tray is narrower than the corresponding portion of the channel in the other tray to provide it with increased retention when coupled to an impression part molded by the other tray.

14. A method for determining a registration coordinate mapping between a human jaw and a volumetric CT (computerized X-ray tomography) image of that jaw, the method comprising:
providing one or more channeled trays made of a rigid material that will not substantially deform when subjected to temperatures of about 70 degrees C., a portion of the surface of the channel in each channeled tray defining a coupling surface region;
providing a moldable thermoplastic material;
inserting the moldable thermoplastic material into a channeled tray comprising a fiducial region in a fixed spatial relation relative to the coupling surface region, the boundary of the fiducial region being detectable in a volumetric CT image;
forming a molded shape of the moldable thermoplastic material in the channeled tray to mate with a surface geometry of at least a portion of the human jaw and the coupling surface region of the channeled tray;
hardening the molded shape of the moldable thermoplastic material into an impression part such that i) when mated to the at least a portion of the human jaw and held within a channel of a channeled tray, the impression part resists displacement relative to the human jaw; ii) the channeled tray is removable from the impression part, iii) each channeled trays of the one or more channeled trays is individually repeatedly attachable to the impression part, and iv) the impression part is removable from, and repeatedly attachable to, the portion of the human jaw;
obtaining a volumetric CT image of a portion of the jaw and the channeled tray coupled to it by the impression part;
removing the channeled tray from the impression part;
bending the impression part away from the surface geometry of the at least a portion of the human jaw to remove the impression part from the human jaw; and subsequently mating the impression part with the surface geometry of the at least a portion of the human jaw; then
coupling a trackable channeled tray that is pose-trackable by the image guidance system to the impression part; and
determining a registration coordinate mapping between the human jaw and its volumetric CT image based on a coordinate mapping between a coordinate frame of the pose-trackable tray and a coordinate frame of the fiducial region's appearance in the image.

15. The method as defined in claim 14 further comprising providing calibration data defining a mapping between a coordinate frame of the trackable channeled tray and a coordinate frame of the fiducial region, and wherein determining the registration coordinate mapping between the human jaw and its volumetric CT image is partly based on the calibration data.

16. The method as defined in claim 14 further comprising determining a selected position in the coordinate frame of the pose-trackable tray, and then using the registration coordinate mapping to map coordinates of the selected position to a corresponding position in the volumetric CT image.

17. The method as defined in claim 14, wherein
each channeled tray defines a corresponding channel comprising a base, a lingual wall and a facial wall, the lingual wall and facial wall extending from the base and spaced from each other so as to be adapted to receive at least one tooth; and
forming the molded shape of the moldable thermoplastic material in the channeled tray comprises placing the channeled tray relative to the jaw such that at least one tooth is received within the corresponding channel of the channeled tray to mold the moldable thermoplastic material therein.

18. The method as defined in claim 17 further comprising molding some of the moldable thermoplastic material over nearby surfaces of the human jaw, the nearby surfaces of the human jaw being outside the corresponding channel of the channeled tray.

19. The method as defined in claim 14, wherein providing the moldable thermoplastic material comprises providing the moldable thermoplastic material as a sheet less than 4 mm in thickness and 10 g in weight

20. The method as defined in claim 14, wherein providing the moldable thermoplastic material comprises providing the moldable thermoplastic material as a sheet less than 2 mm in thickness and 4 g in weight.

21. The method as defined in claim 19, further comprising, before forming the molded shape of the moldable thermoplastic material in the channeled tray, softening the sheet by heating the sheet to a temperature in the range of 50-90° C.

22. The method as defined in claim 19, further comprising, before forming the molded shape of the moldable thermoplastic material in the channeled tray, heating the sheet to a temperature in the range of 50-90° C., to soften the sheet, by blowing air at a temperature higher than 100° C. over a surface the sheet.

23. The method as defined in claim 19, wherein
each channeled tray defines a corresponding channel comprising a base, a lingual wall and a facial wall, the lingual wall and facial wall extending from the base and spaced from each other so as to be adapted to receive at least one tooth; and,
inserting the moldable thermoplastic material into the channeled tray comprises folding or rolling the sheet before inserting into the corresponding channel of the channeled tray.
